(19) United States
(12) Patent Application Publication
(10) Pub. No.: US 2024/0358604 A1
(43) Pub. Date: Oct. 31, 2024

(54) Light Emitting Dental Compositions and Methods of Using the Same

(71) Applicant: Kurt R. Hulse, Onalaska, WI (US)

(72) Inventor: Kurt R. Hulse, Onalaska, WI (US)

(21) Appl. No.: 18/634,419

(22) Filed: Apr. 12, 2024

Related U.S. Application Data
(60) Provisional application No. 63/462,284, filed on Apr. 27, 2023.

(51) Int. Cl. A61K 6/66; A61K 6/853; A61K 6/864; A61K 6/889

(52) U.S. Cl. CPC A61K 6/66; A61K 6/853; A61K 6/864; A61K 6/889

(57) ABSTRACT

Light emitting dental composites are fluorescing composite resins that are produced by combining one or more dental composites, such as, conventional dental composites, glass ionomer cements, resin modified glass ionomers, and/or bioactive dental composites, in either bulk fill form or flowable form, with a light energized and light emitting strontium aluminate or similar compound to form a light emitting dental composite. The light emitting dental composite makes composite resins easily identifiable when activated by a UV light source, allowing for use preferably with temporary placement, visualization, and/or removal of orthodontic attachments and restorative dentistry.

The present invention claims priority to U.S. Prov. Pat. App. No. 63/462,284, titled “Light Energized Flowable Composite,” filed Apr. 27, 2023, which is incorporated herein by reference in its entirety.

### TECHNICAL FIELD

Light emitting dental composites are fluorescing compositions that are produced by combining one or more dental composites, such as, for example, conventional dental composites, glass ionomer cements, a resin modified glass ionomer cement, and/or dental bioactive composites, in either bulk fill form or flowable form, with a light energized and light emitting fluorescing compound, such as, preferably, strontium aluminate or similar compound to form a light emitting dental composition. The light emitting dental composition, which is similar to tooth color in normal light, is easily identifiable when temporarily activated by a UV light source, allowing for use preferably with temporary placement, visualization, and/or removal of orthodontic attachments, sealants, and/or restorative dentistry.

### BACKGROUND

The use of dental cements, specifically conventional dental composites, glass ionomer cement (GIC) and, more specifically, flowable bioactive composites and flowable resin modified glass ionomers (RMGIs), is well-known in dentistry for application of dental and/or orthodontic attachments, and for other dental applications. Generally, a dental composite for use in restorative dentistry and/or for applying attachments and appliances to teeth, comprises a polymer resin matrix and a bulk filler, such as a form of silicate. Conventional dental composites typically comprise of bisphenol A-glycidyl methacrylate and other dimethacrylate monomers, a filler material such as silica and in most applications, a, often, a photoinitiator for curing although curing may also optionally be accomplished through polymerization via two pastes (a base and a catalyst, for example).

A glass ionomer cement is a type of dental composite cement made from a reaction of a silicate glass powder (typically, calciumaluminofluorosilicate glass) and a polyalkenoic acid, such as, for example, polylactic acid, although other glass ionomers may also be formed from other materials. This reaction produces a cement of glass particles surrounded by matrix of fluoride elements and is known chemically as glass polyalkenoate. This dental material has good adhesive bond properties to tooth structure allowing it to form a tight seal between the internal structures of the tooth and the surrounding environment. Therefore, GICs are typically used for filling dental caries as well as to adhere dental and/or orthodontic attachments to tooth enamel.

GICs may be modified to take on other properties to form RMGIs. In one example, GICs can be modified to form UV-curable composites that may be applied to a patient's teeth and rapidly cured via application of a UV light source. Thus, RMGIs are particularly useful for applications where rapid curing is necessary, especially on the attachment of dental and/or orthodontic attachments and/or appliances to teeth.

A relatively new dental composite and/or bonding agent comprises what is known as a “bioactive dental composite,” which are designed to not only replace the missing tooth volume, but also possess therapeutic functions. For example, bioactive dental composites and bonding agents may have remineralizing and antibacterial characteristics that have shown promise in replacing lost minerals, inhibiting recurrent caries, neutralizing acids, repelling proteins, suppressing biofilms and acid production, demonstrating a low cytotoxicity similar to current resins, and protecting dental pulp and promoting tertiary dentin formation.

GICs and RMGIs are particularly useful because they provide cosmetic realism when used for dental applications as they can mimic the color and texture of patient's teeth enamel. Formulations of GICs and RMGIs can be designed in multiple shade variations to better match the color of patients' teeth, thereby being less apparent to the naked eye.

However, because dental composites, such as, for example, GICs and RMGIs, mimic tooth enamel so effectively, they are difficult to distinguish when compared to a patient tooth enamel. The lack of delineation between the artificial composite restoration and the natural enamel produces several complications. Specifically, excess dental composite material can be left behind during placement thereon, such as when used with application of dental and/or orthodontic attachments and/or appliances, which may cause tooth or tissue injury. In addition, loss of dental composite material during treatment may go unnoticed. Further, degradation of virgin tooth enamel may result due to over-reduction of dental composite material during attempted removal thereof.

A need, therefore, exists for an improved dental composite material. Specifically, a need exists for an improved dental composite material that may be easily identifiable to a practitioner during application thereof when used for a dental application. More specifically, a need exists for an improved dental composite material that is easily applied, cured, and maintained during application of the same on a patient's tooth.

Moreover, a need exists for an improved dental composite material that provide easy identification during a dental treatment, which may occur over time. Specifically, a need exists for an improved dental composite material that allows a dental practitioner to distinguish between the composite material and the patient's tooth enamel to identify loss of material during the dental treatment over time.

In addition, a need exists for an improved dental composite material that is easily identifiable and distinguished from a patient's tooth enamel during removal thereof, such as at the end of a dental treatment. Specifically, a need exists for an improved dental composite material that prevents degradation of virgin tooth enamel caused by over-reduction of the composite material during attempted removal thereof.

### SUMMARY OF THE INVENTION

Light emitting dental composites are fluorescing compositions that are produced by combining one or more dental composites, such as, for example, conventional dental composites, glass ionomer cements, resin modified glass ionomers, and/or dental bioactive composites, in either bulk fill form or flowable form, with a light energized and light emitting fluorescing compound, such as, preferably, strontium aluminate or similar compound to form a light emitting dental composition. The light emitting dental composition is easily identifiable, allowing for use preferably with temporary placement, visualization, and/or removal of orthodontic attachments, sealants, and/or restorative dentistry.

To this end, in an embodiment of the present invention, a composition is provided. The composition comprises: a glass ionomer cement; and a fluorescing compound.

In an embodiment, the glass ionomer cement comprises a silicate and a polyalkenoic acid.

In an embodiment, the silicate is calciumaluminofluorosilicate.

In an embodiment, the polyalkenoic acid is polylactic acid.

In an embodiment, the silicate is calciumaluminofluorosilicate and the polyalkenoic acid is polylactic acid.

In an embodiment, the fluorescing compound emits visible light when energized with a UV light having a wavelength of between about 200 nm and about 520 nm.

In an embodiment, the fluorescing compound emits visible light when energized with a UV light having a wavelength of between about 395 nm and about 405 nm.

In an embodiment, the fluorescing compound is strontium aluminate.

In an embodiment, the composition further comprises greater than about 15% by volume of the fluorescing compound.

In an embodiment, the composition further comprises between about 15% by volume and about 40%% by volume of the fluorescing compound.

In an embodiment, the glass ionomer cement is a resin modified glass ionomer cement.

In an embodiment, the resin modified glass ionomer cement comprises a UV curable resin.

In an embodiment, the UV curable resin cures when energized with a UV light having a wavelength of between about 200 nm and about 700 nm.

In an alternate embodiment of the present invention, a method of adhering a dental or orthodontic attachment or appliance to a tooth is provided. The method comprises the steps of providing the composition; dispensing the composition onto a patient's tooth; attaching a dental or orthodontic attachment or appliance to the composition; and curing the composition to adhere the dental or orthodontic attachment or appliance to the patient's tooth.

In an embodiment, the step of curing the composition comprises exposing the composition to a UV light.

In an embodiment, the method further comprises the step of energizing the composition with a UV light so that the composition emits a visible light.

In an embodiment, the UV light has a wavelength of between about 200 nm and about 520 nm.

In an embodiment, the UV light has a wavelength of between about 395 nm and about 405 nm.

In an embodiment, the glass ionomer cement is a modified with a UV curable resin.

In an embodiment, the fluorescing compound comprises strontium aluminate.

It is, therefore, an advantage and objective of the present invention to provide an improved dental composite material.

Specifically, it is an advantage and objective of the present invention to provide an improved dental composite material that may be easily identifiable to a practitioner during application thereof when used for a dental application.

More specifically, it is an advantage and objective of the present invention to provide an improved dental composite material that is easily applied, cured, and maintained during application of the same on a patient's tooth.

Moreover, it is an advantage and objective of the present invention to provide an improved dental composite material that provide easy identification during a dental treatment, which may occur over time.

Specifically, it is an advantage and objective of the present invention to provide an improved dental composite material that allows a dental practitioner to distinguish between the composite material and the patient's tooth enamel to identify loss of material during the dental treatment over time.

In addition, it is an advantage and objective of the present invention to provide an improved dental composite material that is easily identifiable and distinguished from a patient's tooth enamel during removal thereof, such as at the end of a dental treatment.

Specifically, it is an advantage and objective of the present invention to provide an improved dental composite material that prevents degradation of virgin tooth enamel caused by over reduction of the composite material during attempted removal thereof.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

Light emitting dental composites are fluorescing composite resins that are produced by combining one or more dental composites, such as, for example, conventional dental composites, glass ionomer cements, resin modified glass ionomers, and/or bioactive dental composites, in either bulk fill form or flowable form, with a light energized and light emitting strontium aluminate or similar compound to form a light emitting dental composite. The light emitting dental composite make composite resins easily identifiable, allowing for use preferably with temporary placement, visualization, and/or removal of orthodontic attachments and restorative dentistry.

In an embodiment of the present invention, a dental composite is combined with a fluorescing compound to form a light emitting dental composite. The dental composite may be one or more of a conventional dental composite, a glass ionomer cement (GIC), a resin modified glass ionomer cement (RMGI), a bioactive dental composite, or other like dental composite known to one of ordinary skill in the art. Preferably, the dental composite is a GIC and/or an RMGI.

Generally, a glass ionomer cement comprises a reaction product of a basic silicate and a polyalkenoic acid liquid, that sets through an acid base reaction to form a cementitious compound that is useful for dental applications. The acidic polymer is an ionomer, typically comprising an amount of ionic groups, such as, for example, between about 5% and about 10% ionic groups. Preferably, the polyalkenoic acid is polylactic acid. Moreover, in a preferred embodiment, the basic silicate is a calciumaluminofluorosilicate which upon reaction with the polyalkenoic acid, such as polylactic acid, gives a glass polyalkenoate-glass residue set in an ionised, polycarboxylate matrix.

An RMGI is a glass ionomer cement that is modified with a resin modifier. More preferably, the resin modifier within the RMGI typically comprises a resin curative agent that may cure upon application of a UV light, such as, for example, UV light having a wavelength range of between 200 nm and about 700 nm, as is known to one of ordinary skill in the art. Preferable dental cements useful for the light emitting dental composites of the present invention may include Kerr Nexus GIC, Shofu RMGI, and Shofu Beautiful Flow, for example, although the present invention should not be limited as described herein.

Blended with the dental composite, such as the GIC, or, preferably, the RMGI, may be a fluorescing compound or a blend of fluorescing compounds to form a fluorescing or light emitting composition. The fluorescing compound may be activated under, typically, UV light wavelengths to produce emitted light having colors that may be visible and distinctive compared to surrounding tooth enamel. In a preferred embodiment, the dental composite may be a GIC and/or an RMGI wherein the fluorescing compound or compounds may be blended therein. Most preferably, the RMGI may comprise a UV curable resin that may provide rapid curing when applied to a patients tooth for holding dental and/or orthodontic attachments and/or appliances and a fluorescing compound or compounds therein.

Specifically, the fluorescing light emitting dental composition of the present invention may be bonded to enamel and dentin tooth structure using conventional etch/bond/cure techniques, including but not limited to 37% phosphoric acid etching, liquid resin adhesive, and/or the afore-mentioned 200 nm to 700 nm UV light curing to activate bonding to enhance the bonding of the fluorescing glass ionomer cement, or otherwise using the fluorescing glass ionomer cement's natural ability to bond with enamel and dentin through chemical chelation between calcium particles found in the hydroxyapatite crystals of the tooth and the carboxyl groups of the polyalkenoic acid, such as, for example, polylactic acid.

Preferably, the fluorescing compound may be an amount of strontium aluminate powder (SrAl₂O₄) that may be activated under a specific range of light wavelengths to glow a bright color. For example, the specific range of light wavelengths may be between about 200 nm and about 520 nm to produce colors ranging from yellow to purple. Alternatively, the specific range of light wavelengths may be between about 395 nm and about 405 nm to form a green emitted light therefrom. Therefore, the fluorescing dental composite of the present invention may appear normal under typical ambient light conditions but may fluoresce when energized by the specific wavelengths so as to be easily identifiable when energized by the specific wavelengths compared to the patients teeth. Preferable fluorescing compounds from Techno Glow, Inc. comprise strontium aluminate particles are non-encapsulated and are about 50 microns in size, which are similar in proportional size to silica particles used for resin bonding of the dental composites.

Preferably, the fluorescing compound or compounds may be blended with the dental composite, such as the GIC and/or the RMGI, in an amount of more than about 15% by volume. More preferably, the fluorescing compound or compounds may be blended with the dental composite in an amount of between about 15% by volume and about 40% by volume. Most preferably, the fluorescing compound or compounds may be blended with the dental composite in an amount of between about 20% by volume and about 33% by volume.

In a preferred example of the present invention, the fluorescing glass ionomer cement may be a UV curable RMGI, such as Shofu RMGI, blended with between about 15% by volume and about 40% by volume, preferably between about 20% by volume and about 33% by volume strontium aluminate powder, such as strontium aluminate powder manufactured by Techno Glow, Inc., forming a composite in either a bulk fill form or a flowable form to form a fluorescing RMGI of the present invention. The compound may be utilized in dental applications such as, for example, in attaching dental and/or orthodontic attachments and/or appliances to a patient's tooth, as a sealant, and in corrective restoration applications.

Upon application, the practitioner may cure the fluorescing RMGI with an amount of UV radiation. The practitioner may further use an alternate light source, such as, for example, a 3 watt UV LED having a wavelength of between about 395 nm and about 405 nm, to energize the fluorescing RMGI to cause the fluorescing RMGI to fluoresce so that the practitioner may easily identify where the fluorescing RMGI has been applied.

Moreover, the light source for energizing the fluorescing RMGI may be utilized throughout the treatment over time to ensure that the fluorescing RMGI does not become damaged, worn, and/or lose its adherence properties. In addition, upon completion of the treatment, such as when dental and/or orthodontic attachments and/or appliances are removed from the patient's teeth, the light source may be utilized for identifying where the fluorescing RMGI is present for removal therefrom. The application of dental and/or orthodontic attachments and/or appliances or in other applications may be accomplished more effectively without wasting or over applying the fluorescing RMGI or, alternatively, under applying the fluorescing RMGI. Likewise, the dental and/or orthodontic attachments and/or appliances may be removed at the end of treatment by precisely identifying the location of the fluorescing RMGI for removal therefrom, without accidental removal of the patient's enamel.

Generally, the use of fluorescing GIC, such as fluorescing RMGI of the present invention allows for easy verification of placement and position of the composition, allows for easy identification of mistakes and excess material, allows for easy identification of missing attachments. Moreover, compositions of the present invention therefore protects patients' enamel, improves patients' experiences, and improves dental practitioners' accountability.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A light emitting dental composition comprising:
a dental composite; and
a fluorescing compound.

2. The composition of claim 1 wherein the dental composite is selected from the group of a conventional dental composite, a glass ionomer cement, a resin modified glass ionomer, a bioactive dental composite, and blends thereof.

3. The composition of claim 1 wherein the dental composite is a glass ionomer cement comprising a silicate and a polyalkenoic acid.

4. The composition of claim 1 wherein the dental composite comprises a silicate.

5. The composition of claim 4 wherein the silicate is calciumaluminofluorosilicate.

6. The composition of claim 1 wherein the fluorescing compound emits visible light when energized with a UV light having a wavelength of between about 200 nm and about 520 nm.

7. The composition of claim 1 wherein the fluorescing compound emits visible light when energized with a UV light having a wavelength of between about 395 nm and about 405 nm.

8. The composition of claim 1 wherein the fluorescing compound is strontium aluminate.

9. The composition of claim 1 further comprising:
between about 15% by weight and about 40% by weight of the fluorescing compound.

10. The composition of claim 1 further comprising:
between about 20% by weight and about 33% by weight of the fluorescing compound.

11. The composition of claim 1 wherein the dental composite is a resin modified glass ionomer cement.

12. The composition of claim 11 wherein the resin modified glass ionomer cement comprises a UV curable resin.

13. The composition of claim 12 wherein the UV curable resin cures when energized with a UV light having a wavelength of between about 200 nm and about 700 nm.

14. A method of adhering a dental or orthodontic attachment or appliance to a tooth comprising the steps of:
providing the composition of claim 1;
dispensing the composition onto a patient's tooth;
attaching a dental or orthodontic attachment or appliance to the composition; and
curing the composition to adhere the dental or orthodontic attachment or appliance to the patient's tooth.

15. The method of claim 14 wherein the step of curing the composition comprises exposing the composition to a UV light.

16. The method of claim 14 further comprising the step of:
energizing the composition with a UV light so that the composition emits a visible light.

17. The method of claim 16 wherein the UV light has a wavelength of between about 200 nm and about 520 nm.

18. The method of claim 16 wherein the UV light has a wavelength of between about 395 nm and about 405 nm.

19. The method of claim 14 wherein the dental composite is a resin modified glass ionomer comprising a UV curable resin.

20. The method of claim 14 wherein the fluorescing compound comprises strontium aluminate.
